(19) United States
(12) Patent Application Publication
(10) Pub. No.: US 2009/0125051 A1
(43) Pub. Date: May 14, 2009

(54) COMBINATION COIL AND LIQUID EMBOLIC FOR EMBOLIZATION

(75) Inventors: John O'Gara, Ashland, MA (US); Robert E. Richard, Wrentham, MA (US)

Correspondence Address: VISTA IP LAW GROUP LLP, 12930 Saratoga Avenue, Suite D-2, Saratoga, CA, 95070, US

(73) Assignee: BOSTON SCIENTIFIC SCIMED, INC., Maple Grove, MN (US)

(21) Appl. No.: 12/268,337

(22) Filed: Nov. 10, 2008

Related U.S. Application Data
(60) Provisional application No. 60/987,702, filed on Nov. 13, 2007.

(51) Int. Cl. A61F 2/00

(52) U.S. Cl. 606/191

(57) ABSTRACT

A system and method for treating a target site (e.g., vascular aneurysm), the system including an occlusive member (e.g., a coil) configured to be positioned within the target site, the occlusive member comprising a first reactant disposed thereon. The system further includes a second reactant that is introduced within the target site in close proximity to the occlusive member (e.g., using a same delivery device used to deliver the occlusive member to the target site), wherein a polymer filling may be formed by reacting the first reactant with the second reactant, the polymer filling helping to anchor the occlusive member within the target site. In one embodiment, the first reactant includes a prepolymer while the second reactant includes an activator.

### CROSS-REFERENCE TO RELATED APPLICATIONS

The present application claims the benefit under 35 U.S.C. §119 to U.S. Provisional Application No. 60/987,702 filed Nov. 13, 2007, the contents of which are incorporated herein by reference as though set forth in full.

### FIELD OF THE INVENTION

The present invention is in the field of medical and methods for embolizing blood vessels using a combination of an occlusive device, such as a coil, and a polymeric glue or glue-like media.

### BACKGROUND

Embolization of blood vessels is conducted for a variety of purposes including the treatment of tumors, the treatment of lesions such as aneurysms, arteriovenous malformations (AVM), arteriovenous fistula (AVF), uncontrolled bleeding and the like. Several devices and methods are known in the art for embolizing blood vessels, for example, those disclosed in U.S. Pat. No. 5,702,361 to Evans et al., U.S. Pat. No. 5,891,192 to Murayama et al., U.S. Pat. No. 6,015,541 to Greff et al., and U.S. Pat. No. 5,202,352, all of which are incorporated by reference herein in their entirety.

Coils and liquid embolics have been used for embolization procedures in both interventional neuroradiology and peripheral vascular applications. However, liquid embolics or glues suffer from drawbacks. Incorrect mixing can lead to embolization at an undesired site. Care must also be taken to ensure that the glue does not harden in the catheter or that catheter does not become glued to the treatment area. Coiling may also suffer from several drawbacks including aneurysm perforation, improper coil position, vasospasm, and partial artery occlusion. Coated coils are known to address some of these problems, specifically improved filling and occlusion as well as improved healing properties, as discussed, for example in Bui, J. T.; West, D. L.; Pai, R.; Owens, C. A. Cardiovasc. Intervent. Radiol. 2006, 29, 1121-1124, which is incorporated by reference herein in its entirety. Liquid embolics used in conjunction with coils is being investigated to improve procedural outcomes, but for the aforementioned reasons, the drawbacks are compounded by the two approaches as opposed to decreased. There thus is a need for improved materials and methods that combine the positive features of liquid embolics and coils without their respective drawbacks.

### SUMMARY

In one embodiment, a method of treating a target site or location (e.g., vascular aneurysm) includes inserting an occlusive member such as a coil within the aneurysm, the occlusive member includes a first reactant disposed thereon. A second reactant is introduced within the aneurysm and in close proximity to the occlusive member. A polymer filling is formed by reacting the first reactant with the second reactant. In one embodiment, the first reactant includes a prepolymer while the second reactant includes an activator. The polymer filling may serve to anchor the occlusive member at the target location.

In another embodiment, a system for occluding a target location (e.g., aneurysm) includes an occlusive member having a first reactant disposed thereon and a second reactant configured to react with the first reactant to form a polymer filling. The first and second reactants only react in each other's presence and are otherwise innocuous. The system includes a delivery member for delivering the second reactant at or adjacent to the occlusive member. The second reactant may be delivered via a delivery catheter which may be the same delivery catheter that delivers the occlusive member or, alternatively, it may be a different delivery catheter.

### BRIEF DESCRIPTION OF THE DRAWINGS

FIG. 1 illustrates a side view of an occlusive member according to one aspect of the invention.

FIG. 2 illustrates a cross-sectional view of an occlusive member according to another aspect of the invention.

FIG. 3 illustrates a side view of an occlusive member according to another aspect of the invention. This embodiment includes a fiber disposed on the occlusive member.

FIG. 4 illustrates a side view of an occlusive member according to another aspect of the invention. This embodiment illustrates an open pitch configuration which forms a gap or interstitial space between adjacent windings of the occlusive member.

FIG. 5 illustrates a partial cross-sectional view of an aneurysm illustrating delivery of an occlusive member according to one embodiment of the invention.

FIG. 6 illustrates a partial cross-sectional view of an aneurysm illustrating delivery of the second reactant to the occlusive member so as to initiate the formation of the polymer anchor.

FIG. 7 illustrates a perspective view of an occlusive member delivery system. The occlusive member is shown with incorporated fibers.

### DETAILED DESCRIPTION OF THE ILLUSTRATED EMBODIMENTS

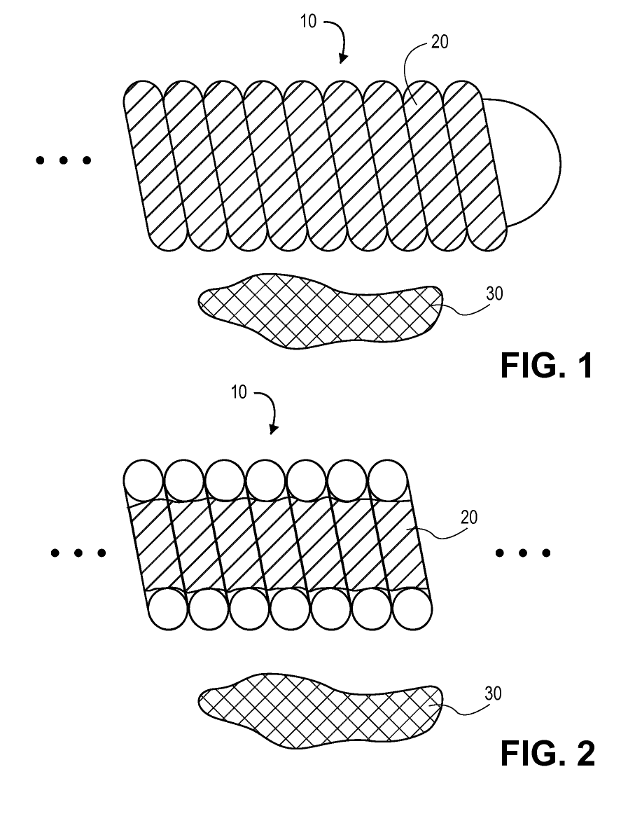

Embodiments of the present invention are directed to a combination of an occlusive member and liquid embolic material for embolization. FIGS. 1-7 illustrate an occlusive member 10 in the form of a coil according to various aspects of the invention. Of course, an occlusive member 10 may include other devices and shapes beyond the illustrated coil. A typical occlusive member 10 in the form of a coil may be formed by winding a platinum wire strand about a primary mandrel and applying a heat treatment to impart a primary winding coil shape. The relative stiffness of the occlusive member 10 will depend, among other things, on the diameter of the wire strand, the diameter of the primary mandrel, and the pitch of the primary windings. The device is then wrapped around a secondary mandrel, and again heat treated to impart a secondary shape. For example, U.S. Pat. No. 4,994,069, to Ritchart et al., describes a vaso-occlusive coil that assumes a primary, linear helical configuration when stretched and a folded, and a convoluted, secondary configuration when relaxed in a minimal energy configuration. The stretched condition is used in placing the coil at the desired site (by its passage through a delivery catheter) and the coil assumes a relaxed configuration—which is better suited to occlude the vessel—once the device is so placed.

The diameter of the wire used in the production of the coils 10 may fall in the range of about 0.00025 inches to about 0.006 inches. The coil 10 may have a primary diameter of between about 0.003 and about 0.025 inches, but for most neurovascular applications, a diameter between about 0.008 to about 0.018 inches provides sufficient hoop strength to hold the coil 10 in place within the chosen body site, lumen, or cavity, without substantially distending the wall of the site and without moving from the site as a result of the repetitive fluid pulsing found in the vascular system.

The axial length of the coil wire will usually fall in the range of around 0.5 to around 100 cm, more usually around 2.0 to 40 cm. Of course, all of the dimensions provided above should be viewed only as guidelines, and the invention, in its broader aspects, should not be limited thereto. Dimensions that are suitable for use in occluding sites within the human body are included in the scope of this invention.

While the occlusive members 10 that are used with the methods described herein are normally made of biocompatible metals such as platinum, gold, tungsten, titanium, tantalum, and the like or alloys of such metals, the bodies can also be made of bioabsorbable or nonbioabsorbable polymers or copolymers. Examples of bioabsorbable polymers that have been used to make intralumenal implants are polyglycolic acid, polyglycolic/poly-L-lactic acid copolymers, polyortheosters, polycaprolactive, polyhydroxybutyrate/hydroxyvalerate copolymers, poly-L-lactide, polydioxanone, polycarbonates, “pseudo” polyamino acids (amino acid polymers in which peptide bonds have been replaced with other linkage groups) and polyanhydrides. Examples of nonbioabsorbable polymers that have been used to make intralumenal implant bodies are polyethylene terephthalate, polyurethane urea, and silicone polymers. Other bioabsorbable and nonbioabsorbable polymers that may be used to make intralumenal implants are described in U.S. Pat. No. 5,527,337; EPA Pub. No. 0604022A1, PCT Pub. No. WO 93/15787; “Biodegradable Stents”, Zidar, J. P. et al., Textbook of International Cardiology, 2nd Edition, W. B. Saunders Company (1994) pp. 787-802; “Current Status of Biodegradable Stents”, Tanguay, J. F. et al., Cardiology Clinics, Vol. 12, No. 4, W. B. Saunders Company (1994) pp. 699-713; Langer, R., Annals of Biomedical Engineering (1995) 23:101-111; and Pulapura, S. et al., J. Biomater. Appl. (1992) 6(3):216-250, all of which are incorporated by reference herein in their entirety.

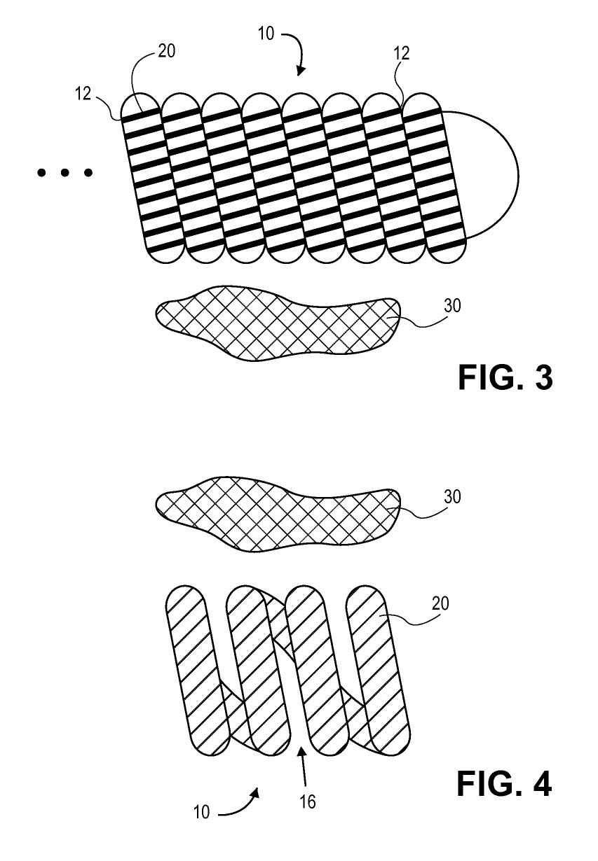

In some embodiments, for example, as illustrated in FIGS. 3 and 7, the coil 10 further comprises fiber 12. In some embodiments, the fiber 12 covers all or a portion of the coil 10. Alternatively, strands of fiber 12 can be wound around the coil 10. In other alternative embodiments, the coil 10 comprises tufts of fiber 12 or fiber bundles attached to it, so as to increase the amount and volume of fiber held by the coil 10. The fibers 12 may be closely associated with the exterior of the coil 10 as is illustrated in FIG. 3 or they may be attached at one end with remaining ends free from the coil 10. Fibered vaso-occlusive devices have been described in the art. Vaso-occlusive coils having attached fibers are shown in U.S. Pat. Nos. 5,226,911 and 5,304,194, both to Chee et al. Another vaso-occlusive coil having attached fibrous materials is found in U.S. Pat. No. 5,382,259, to Phelps et al. The Phelps et al. patent describes a vaso-occlusive coil which is covered with a polymeric fibrous braid on its exterior surface. U.S. Pat. No. 5,658,308, to Snyder, is directed to a vaso-occlusive coil having a bioactive core. All of the above patents are incorporated by reference herein in their entirety.

Liquid embolics are effective for their treatment purposes when they polymerize at the site of treatment, e.g., a vascular aneurysm. The drawbacks discussed above associated with the use of liquid embolics arise when the polymerization occurs at a site other than the intended treatment site, or that the polymerization causes the catheter to adhere to a tissue, such as a blood vessel. To overcome the drawbacks of conventional use of coils and liquid embolics, the methods described herein are designed to anchor the polymer resulting from the liquid embolic onto the coil 10 which can reduce coil movement or slippage. In addition, the polymer filling results in improved occlusion of the target site. In addition, by having targeted polymer formation on the coil 10, this helps in preventing the delivery member (e.g., catheter) from inadvertently getting stuck or adhered. This is achieved by utilizing a multi-component reactant system to ensure that polymerization takes place in close proximity to the coil 10. The terms “close proximity” is meant to include at the coil 10 or adjacent or substantially adjacent to the coil 10.

In accordance with one aspect of the invention, the coil 10 is loaded with a first reactant 20. FIG. 1, for example, illustrates a first reactant 20 disposed on the exterior surface of the coil 10. The first reactant 20 may be applied to the exterior of the coil 10 as a film, coating, or it may be integrated into the coil 10 itself. The first reactant 20 may also be populated only a portion or portions of the coil 10. Alternatively, as shown in FIG. 2, the interior portion 14 of the coil 10 may be loaded with the first reactant 20. In still another alternative, as illustrated in FIG. 3, the first reactant 20 may be coated or integrated into the fibers 12. While FIGS. 1-3 illustrate a coil 10 having adjacent coil segments substantially close together (e.g., closed pitch), in still other configurations such as that illustrated in FIG. 4, the coil 10 has a an open pitch with gaps or interstitial spaces 16 between adjacent coil windings. The gaps or interstitial spaces 16 created within the coil 10 can then be filled in with the glue or adhesive material formed by the reaction of the first reactant 20 and the second reactant 30.

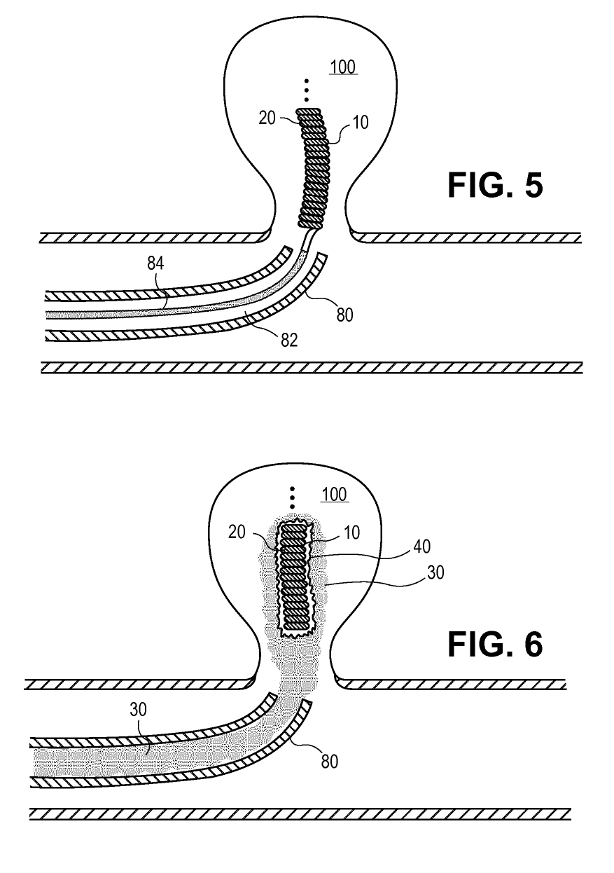

The coil 10 is then placed in the treatment site, for example an aneurysm, in a conventional way known in the art. FIG. 5 illustrates a coil 10 that is placed within an aneurysm 100. This process typically involves a delivery member 80 (which may take the form of a delivery catheter) that is advanced adjacent to or even within the aneurysm 100. The coil 10 is advanced down a lumen 82 of the delivery catheter 80 via a pusher wire 84 or the like. The coil 10 is then detached from pusher wire 84 using any number of detachment modalities. These include, for example, mechanical, thermal, and electrolytic detachment schemes. U.S. Pat. No. 5,122,136 issued to Guglielmi et al. discloses an electrolytic detachment modality. U.S. Pat. No. 6,966,892 issued to Ganshi et al. discloses a thermal detachment modality. U.S. Pat. No. 5,800,453 issued to Gia discloses a mechanical detachment system. The above-noted U.S. Patents are incorporated by reference as if set forth fully herein.

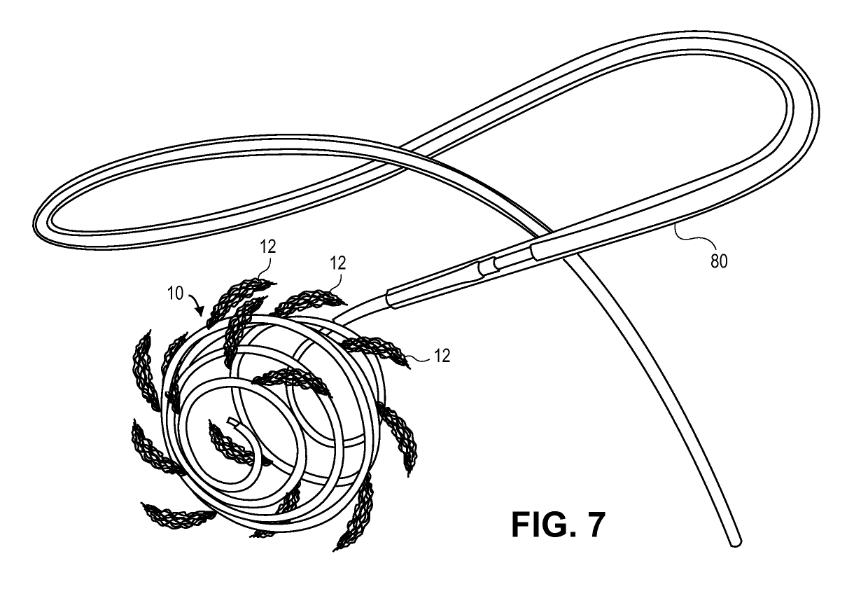

Referring now to FIG. 6, a liquid comprising a second reactant 30 is injected into the treatment area 100. The injection step may be simultaneous with the placement of the coil 10 in the treatment area 100, or alternatively, the injection step occurs subsequent to the placement of the coil 10 in the treatment area 100. The second reactant 30 may be delivered via the same delivery catheter 80 that is used to deploy the coil 100. Alternatively, a different delivery catheter 80 may be used to deliver the second reactant 30. For example, the second reactant 30 may be injected via the lumen 82 into the treatment area 100. A proximally located syringe or the like (not shown) may be used to forcibly deliver the second reactant 30 to the treatment area 100. As seen in FIG. 6, as the first and second reactants 20, 30 come into contact with each other, a polymerization reaction takes place which causes formation of the final polymeric filling material 40. The polymeric filling material 40 may be physically entrapped or entrained in and/or around the coil 10. Alternatively, the polymeric filling material 40 may be chemically bonded in and/or around the coil 10 and serves to anchor the coil 10.

In still another aspect, one of the first reactant 20 or the second reactant 30 may be delivered to the target site followed by delivery of the coil 10. The coil 10 may be pre-loaded with the other of the first reactant 20 or second reactant 30. When the coil 10 contacts the reactant 20, 30, the entire mass hardens to occlude the target site.

In one embodiment, the first reactant 20 is a prepolymer, e.g., a monomer or a mixture of copolymers, and the second reactant 30 is a polymerization initiator, also referred to as an activator. Alternatively, the second reactant 30 may include a prepolymer while the first reactant 20 is an activator. In this regard, the first reactant 20 may be disposed on the coil 10 or an associated structure (e.g., fibers 12) while the second reactant 30 is delivered to the treatment area 100. Alternatively, the second reactant 30 may be disposed on the coil 10 or associated structure while the first reactant 20 is delivered to the treatment area 100. Activators, or polymerization initiators, begin the process of polymerization by reacting with one molecule of a monomer. Once the monomer reacts with the activator, the combination of the monomer/activator acts as a new activator and reacts with another molecule of the monomer. The polymerization chain reaction continues in this manner until the chain reacts with a compound that is designed not to propagate the chain further, and the polymerization is terminated. Generally, the activator is a present in a lesser amount (wt/wt) than the prepolymer. Of course, for other systems the relative amounts may be substantially equal.

In some cases, the chain reacts with the activator again and a branch in the chain is created. In other cases, one of the monomers can react with two or more different chains at different sites on the monomer. In these cases, the two or more polymeric chains join together at one point and form a cross-linked polymer. The cross-linked polymers show greater strength and stability than single chain polymers. In other embodiments, however, single chain polymers may be created. Single chain polymers generally exhibit greater flexibility than cross-linked polymers.

In one embodiment, the first reactant 20 may elute from the coil 10 and initiate a reaction with the second reactant 30 to form a polymeric filling 40 within the interstitial zones 16 of the coil 10. Alternatively, the first reactant 20 remains on the coil 10, and the second reactant 30 comes into contact with the first reactant to form the polymeric filling 40. Of course, as described above, the second reactant 30 may be disposed on the coil 10 or other associated structure and elute from the coil 10 and initiate reaction with the first reactant 20 that is delivered to the treatment area 100.

One of the first and second reactants 20, 30 may optionally be loaded onto the fiber 12 if such a structure is integrated into or on the coil 10. For instance, the first or second reactant 20, 30 may react with the fiber 12 so that it becomes bound thereto and does not leach out of the fiber 12. Alternatively, the fiber 12 just holds or retains the first or second reactant 20, 30 and upon placement of the coil 10 into the treatment area 100, the first or second reactant 20, 30 can leach out of the fiber 12.

The polymeric filling 40, which results from the reaction of the first and second reactants 20, 30 can be completely, or partially, within the interstitial zone 16 of the coil 10. For example, in the embodiment of FIG. 4, the polymeric filling material 40 may fill the interstitial zone 16 to aid in anchoring the deployed coil 10 in place. The polymeric filling 40 may also be physically entrapped within the interior or lumen of the coil 10, and in some instances, can be bonded to the coil 10 or the surface of the fiber 12.

The polymeric filling 40 may be formed from a biostable material. In the context of the present disclosure, “biostable” means that a particular compound or polymer does not degrade under physiological conditions, or that the degradation rate is slow, for example having a half-life on the order of years. For example, a biostable polymer does not dissolve in water or other physiological fluids and does not get metabolized by enzymes commonly present in physiological fluids.

Alternatively, the polymeric filling 40 may be biodegradable. In the context of the present disclosure, “biodegradable” means that a particular compound or polymer has a relatively fast degradation rate, for example having a half-life on the order of weeks or months. For example, a biodegradable polymer dissolves in water or other physiological fluids or is metabolized by enzymes commonly present in physiological fluids.

It should be understood that, by themselves, the first and second reactants 20, 30 do not self-polymerize. The polymeric filling 40 is formed only when the first and second reactants 20, 30 are brought in contact to each other. In one aspect, both the first and second reactants 20, 30 are non-toxic and, in the absence of polymerization, are either metabolized by the body or are excreted therefrom, for example through the kidneys or the liver. Also, when the first and second reactants 20, 30 are brought in contact with each other, in some instances not all of the one reactant 20, 30 reacts with the other reactant 20, 30. The un-reacted molecules of the reactants 20, 30 may then metabolized by, or excreted from, the body. In some embodiments, the first or second reactant may carry with it one or more therapeutic agents, e.g., an anti-inflammatory agent, an anti-microbial agent, or a chemotherapeutic agent.

The first and second reactants 20, 30 may be chosen such that they react with each other by a variety of different mechanisms. The filling polymer 40 can be polymerized by an ionic cross-linking reaction, where copolymers having α,β-unsaturated carboxyl groups react with a water-soluble metal salt to form the polymer. An example of ionic cross-linking reactions and mechanism is found in U.S. Pat. No. 5,003,001, or Skaugrud et al., Biotechnology and Genetic Engineering Reviews, 1999, 16, 23-40, both of which incorporated by reference herein in their entirety.

Nucleophilic reactions are useful polymer-forming reactions. Michael-type reactions, where a nucleophile, for example an enolate anion, reacts with an electron-poor olefin, for example an α,β-unsaturated group, in conjugate additions, can also be used to form the filling polymer 40. Variations on the traditional Michael-type reactions, for example by using thiocarboxyl groups or thiolate anions, can also be used. The filling polymer 40 may also include hydrogels formed by hydrolytically labile poly(ethylene glycol)-based hydrogels formed via Michael-type addition reactions between unsaturated acrylate moieties and nucleophilic thiols. Examples of such hydrogels may be found in Metters et al., Network Formation and Degradation Behavior of Hydrogels Formed by Michael-Type Addition Reactions, Biomacromolecules 2005, 6, 290-301, which is incorporated by reference as if set forth fully herein. Well-known urea or urethane polymer chemistry, for example, by reacting amine substituted or alcohol substituted molecules with isocyanates, can be used.

Radical initiated reactions are well-known in polymer chemistry. The activator in these reactions can be a radical initiator while the prepolymer can be a vinyl monomer. Examples of initiators include, but are not limited to azo compounds, peroxides, disulfides, inorganic and organic peroxide systems, and the like. The radical can be generated chemically or by radiation, such as UV radiation. In some embodiments, when UV radiation is used to initiate the radical formation, the catheter 80 used to transport the occlusive member 10 into the treatment site also comprises an optic path, such as a fiber optic line, for the UV light to reach the treatment site.

Examples of compounds that can be used as prepolymers with the methods disclosed herein include, but are not limited to, sodium alginates, acrylates, acrylamides, maleimides, vinyl sulfones, quinones, vinyl pyridinium, poly(ethylene glycol) diacrylate and/or combinations thereof. Examples of compounds that can be used as activators with the methods disclosed herein include, but are not limited to, divalent salts of calcium, barium, and strontium (i.e., Ca⁺², Ba⁺², and Sr⁺²), thiols, amines, alcohols, 1,4-dimercapto-2,3-butanediol, pentaerythrithiol and/or combinations thereof.

It should be understood that the methods disclosed herein can be used with any of the occlusion coils 10 known in the art. Examples of such coils 10, without limitation, are those described in U.S. Pat. Nos. 4,994,069, 5,122,136, 5,599,326, 5,582,619, 5,624,461, 5,549,624, and 5,304,194, all of which are incorporated by reference herein in their entirety.

What is claimed is:

1. A method of treating a target location in a body, comprising:
inserting an occlusive member into the target location, the occlusive member comprising a first reactant disposed thereon;
introducing a second reactant into the target location and in close proximity to the occlusive member, wherein a polymer filling is formed by reaction of the first reactant with the second reactant, wherein the polymer filling serves to anchor the occlusive member at the target location.

2. The method of claim 1, wherein the target location is selected from the group consisting of an aneurysm, a tumor, a lesion, and a site of uncontrolled internal bleeding.

3. The method of claim 1, one of the first reactant and the second reactant comprising prepolymer, and the other of the first reactant and second reactant comprising an activator.

4. The method of claim 1, the occlusive member having at least one fiber secured thereto.

5. The method of claim 4, wherein one of the first reactant and the second reactant is adhered to the fiber.

6. The method of claim 4, wherein one of the first reactant and the second reactant leaches out of the fiber under exposure to physiological conditions at the target location.

7. The method of claim 3, wherein a polymer anchor is formed by irradiating the activator within the target location with a radical-initiating radiation.

8. The method of claim 3, wherein the activator comprises a radical initiator selected from the group consisting of azo compounds, peroxides, disulfides, inorganic peroxide systems, and organic peroxide systems.

9. The method of claim 3, wherein the activator comprises a nucleophile and the prepolymer is an electron-poor olefin.

10. The method of claim 1, wherein the polymer anchor is formed by an ionic cross-linking reaction, the polymer anchor comprising a straight chain polymer or a cross-linked polymer.

11. The method of claim 1, wherein the occlusive member and the second reactant are delivered to the target location using a same delivery device.

12. A system for occluding a target location in a body, comprising:
an occlusive member having a first reactant disposed thereon;
a second reactant adapted to react with the first reactant, wherein a polymer filling is formed by reaction of the first reactant with the second reactant;
a delivery member configured for delivering one or both of the occlusive member and second reactant to a target location in a body.

13. The system of claim 12, one of the first and second reactants comprising a prepolymer and the other comprising an activator.

14. The system of claim 13, wherein a polymer anchor is formed by irradiating the activator with a radical-initiating radiation in the presence of the prepolymer within the target location.

15. The system of claim 13, wherein the activator comprises a radical initiator selected from the group consisting of azo compounds, peroxides, disulfides, inorganic peroxide systems, and organic peroxide systems.

16. The system of claim 13, wherein the activator comprises a nucleophile and the prepolymer is an electron-poor olefin.

17. The system of claim 13, wherein a polymer anchor is formed by an ionic cross-linking reaction, the polymer anchor comprising a straight chain polymer or a cross-linked polymer.

18. The system of claim 12, further comprising means for irradiating the second reactant with a radical-initiating radiation after the occlusive member and second reactant have been delivered to the target location.

19. The system of claim 12, wherein the delivery member is configured to deliver the occlusive member to the target location, the system further comprising a second delivery member configured to deliver the second reactant to the target location.

20. The system of claim 12, the occlusive member having at least one fiber secured thereto.

21. The system of claim 20, wherein one of the first reactant and the second reactant is adhered to the fiber.

22. The system of claim 20, wherein one of the first reactant and the second reactant is chemically bound to the fiber.

23. The system of claim 20, wherein one of the first reactant and the second reactant leaches out of the fiber under exposure to physiological conditions at the target location.

24. The system of claim 12, wherein the occlusive member comprises a coil.
